(19) United States
(12) Patent Application Publication
(10) Pub. No.: US 2021/0205398 A1
(43) Pub. Date: Jul. 8, 2021

(54) COMPOSITIONS CONTAINING PHRAGMITIS RHIZOMA EXTRACT AS ACTIVE INGREDIENT FOR PREVENTION, AMELIORATION, OR TREATMENT OF A DISORDER CAUSED BY SIDE EFFECT OF ANTICANCER AGENT

(71) Applicant: KOREA INSTITUTE OF ORIENTAL MEDICINE, Daejeon (KR)

(72) Inventors: Jinhee KIM, Daejeon (KR); Eun Sang CHO, Daejeon (KR); No Soo KIM, Daejeon (KR); Ok-Sun BANG, Daejeon (KR); You Jin LEE, Daejeon (KR); Young-Ah KIM, Daegu (KR); Eunna HUH, Gyeonggi-do (KR)

(21) Appl. No.: 15/999,722

(22) PCT Filed: Feb. 17, 2017
(86) PCT No.: PCT/KR2017/001813

§ 371 Date: Aug. 20, 2018

(30) Foreign Application Priority Data
Feb. 18, 2016 (KR) 10-2016-0019339

(51) Int. Cl. A61K 36/899; A61P 7/06; A23L 33/105; A23L 33/00

(52) U.S. Cl. CPC A61K 36/899; A61P 7/06; A23L 33/105; A23V 2002/00; A61K 2236/331; A61K 2236/333; A23L 33/40

(57) ABSTRACT

A composition contains Phragmitis Rhizoma extract as an active ingredient. As a result of treating mouse bone marrow cells with an extract of Phragmitis Rhizoma, it was confirmed to have an effect that a decrease in colony of hematopoietic cells, which is induced by an anticancer agent, is significantly recovered and bone marrow suppression caused by an anticancer agent is significantly recovered in an animal model which has been induced to have bone marrow suppression according to intraperitoneal injection of an anticancer agent. Thus, the Phragmitis Rhizoma extract can be advantageously used for a composition for prevention, amelioration, or treatment of a disorder caused by side effect of an anticancer agent, and there is a possibility of having the increased effect of an anticancer agent when it is used as an anticancer adjuvant in combination with an anticancer agent.

### TECHNICAL FIELD

The present invention relates to a composition containing Phragmitis Rhizoma extract as an active ingredient for prevention, amelioration, or treatment of a disorder caused by side effect of an anticancer agent.

### BACKGROUND ART

Cancer is the first or second most common causes of death in South Korea. Cancer is response for the death of almost 30% of the people who died in their 50s or 60s in South Korea. As a cancer therapy, a surgical operation, radiotherapy, and chemotherapy are most widely employed. In particular, as chemotherapy, many efforts have been made to develop an anticancer agent that completely eradicates most types of cancer, although an anticancer agent working as a true therapeutic agent is yet to be developed. Most chemotherapeutic agents of current medicine just remains as an agent for extending survival for a short period of time.

The anticancer agent used for chemotherapy interrupts metabolic pathways of cancer cells by directly working on DNAs and blocking replication, transcription, or translational process of DNAs, or preventing synthesis of nucleic acid precursors to inhibit cell division, and thus exhibiting cytotoxicity. However, anticancer agents that are currently used have no selectivity for specific cancer, and thus they have a characteristic that the therapeutic effect on cancer cells and toxic effect on normal cells are exhibited simultaneously. Furthermore, the toxicity caused by administration of an anticancer agent includes various side effects such as cytopenia of white blood cells, platelets, red blood cells, or the like resulting from bone marrow failure, hair loss caused by damaged follicle cells, irregular period or male infertility caused by toxic effect exhibited on ovary or testicle, stomatitis, nausea, dysphagia, and celiac disorder as a side effect caused by disrupted mucous membrane cells of digestive system, diarrhea, nephrotoxicity caused by tubular necrosis, peripheral neuritis or fatigue caused by neurological disorder, vascular disorder like angialgia and rash, and discoloration of skin or finger nails and toe nails. Under the circumstances, it is strongly desired to develop a pharmaceutical agent having increased anticancer effect with minimal adverse side effects.

Phragmitis Rhizoma (noh-geun), which is also referred to as we-geun, is dried roots of a reed (Phragmitis commnis Trin) that is a plant belonging to the family Gramineae. Reed is found in marsh, river bank, wetland, or sea shore. During spring or autumn, roots of Phragmitis commnis Trin are collected, fine roots are removed, washed with water, and dried under the sun. Dried Phragmitis Rhizoma has a flat cylinder shape with glossy surface and yellowish white color. Nodes of the roots are somewhat hard and exhibit distinctive yellowish red color. Vertical wrinkles are present between nodes.

Phragmitis Rhizoma has a sweet taste and traditionally it is known to have a cold property and works on lung and stomach meridian. Pharmaceutical effect of Phragmitis Rhizoma includes lowering body heat and stopping nausea by producing phlegm fluid, and it has been used for treating nausea caused by fever, esophagus cancer, or lung abscess, or detoxicating puffer fish toxin. It is also reported to have an activity of blocking ultraviolet rays, causing dark and shiny hair by promoting proliferation of melanocytes, and whitening skin and supplying nutrients to skin when used in a skin whitening cream. As for the study relating to the physiological activity of Phragmitis Rhizoma, there is a report indicating that Phragmitis Rhizoma extract exhibits an influence on glucose, insulin, and lipid synthesis in serum, and it is also reported to have an effect of suppressing the increase of glucagon granules in Langerhans A cells, which is caused by administration of streptozotocin, and suppressing insulin degranulation in Langerhans B cells. It is also reported that methanol extract of Phragmitis Rhizoma has an effect of lowering blood cholesterol level and blood glucose level. It is also reported that methanol extract of Phragmitis Rhizoma root stalk can significantly lower triglyceride concentration in a mouse with hypertriglycemia, hypercholesterolemia, or diabetic hyperlipidemia, and β-sitosterol and p-coumaric acid separated from the methanol extract have an effect of improving serum lipid concentration.

As a technique relating to an extract of Phragmitis Rhizoma, a method for producing an extract with immune-enhancing and anticancer activity from Phragmitis Rhizoma is disclosed in Korean Patent Registration No. 1098875, and a composition for skin miniaturization or treatment of dermatitis or atopic dermatitis containing an extract of Phragmitis Rhizoma as an active ingredient is disclosed in Korean Patent Registration No. 1151718. However, a composition containing Phragmitis Rhizoma extract as an active ingredient for prevention, amelioration, or treatment of a disorder caused by side effects of anticancer agents as described in the present invention has never been disclosed.

### DETAILED DESCRIPTION OF THE INVENTION

### Technical Problems to be Solved

The present invention is devised under the circumstances described above. The present invention relates to a composition containing Phragmitis Rhizoma extract as an active ingredient for prevention, amelioration, or treatment of a disorder caused by side effects of anticancer agents, and more specifically, by confirming that the survival of hematopoietic stem and progenitor cells is significantly restored as the bone marrow cells of a mouse, which has reduced hematopoietic stem and progenitor cells due to administration of an anticancer agent, are treated with the Phragmitis Rhizoma extract of the present invention, and also bone marrow suppression caused by an anticancer agent is significantly recovered in an animal model having bone marrow suppression that is induced by intraperitoneal injection of an anticancer agent, the present invention is completed.

### Technical Means for Solving the Problems

To achieve the purpose described above, the present invention provides a composition containing Phragmitis Rhizoma extract as an active ingredient for prevention or treatment of a disorder caused by side effects of anticancer agents.

The present invention further provides a functional health food containing Phragmitis Rhizoma extract as an active ingredient for prevention or amelioration of a disorder caused by side effects of anticancer agents.

The present invention still further provides an anticancer adjuvant agent containing Phragmitis Rhizoma extract as an active ingredient.

### Advantageous Effect of the Invention

The present invention relates to a composition containing Phragmitis Rhizoma extract as an active ingredient for prevention or treatment of a disorder caused by side effects of anticancer agents. According to the present invention, not only a decrease in hematopoietic stem cell colony caused by an anticancer agent can be significantly restored but also bone marrow suppression caused by an anticancer agent can be significantly recovered so that the Phragmitis Rhizoma extract of the present invention can be used for a pharmaceutical composition, a functional health food, or an anticancer adjuvant agent for prevention or treatment of a disorder caused by side effects of anticancer agents.

### BRIEF DESCRIPTION OF THE DRAWINGS

FIG. 1 shows the result of determining the effect of an anticancer agent (docetaxel, doxorubicin, irinotecan, paclitaxel, or daunorubicin) at different concentrations on growth and differentiation of bone marrow cells which have been isolated from a mouse femur, in which the determination was made based on CFU (colony forming unit) assay. In the figure, *, **, and *** indicate that the treatment with an anticancer agent has a statistically significant difference compared to CFU of the control group which has not been treated with any anticancer agent. * means p value of less than 0.05, ** means p value of less than 0.01, and *** means p value of less than 0.001.

FIG. 2 shows the result of determining the number of bone barrow cells from femur of C5BL/6 mouse after intraperitoneal injection of docetaxel at concentration of 50, 100, or 150 mg/kg, in which the determination was made (A) 3 days or (B) 7 days after the injection. In the figure, ** and *** indicate that the cell survival rate of the docetaxel administration group has a statistically significant difference compared to the control group (Veh) which has not been treated with docetaxel. ** means p value of less than 0.01 and *** means p value of less than 0.001.

FIG. 3 shows the result of determining a decrease in the number of mouse bone marrow cells caused by a treatment with an anticancer agent (docetaxel, doxorubicin, irinotecan, paclitaxel, or daunorubicin), and the effect of restoring the decrease in the number of mouse bone marrow cells according to a combined treatment of the anticancer agent with the Phragmitis Rhizoma extract of the present invention at concentration of 25, 50, or 100 μg/ml. ##, ###, and #### indicate that the CFU obtained from treatment with an anticancer agent has a statistically significant difference compared to the control group which has not been treated with any anticancer agent, in which ## means p value of less than 0.01, ### means p value of less than 0.001, and #### means p value of less than 0.0001. *, **, and *** indicate that there is a statistically significant difference between the CFU obtained from the group which has been treated with an anticancer agent and the CFU obtained from the group which has been treated with an anticancer agent and the Phragmitis Rhizoma extract in combination, in which * means p value of less than 0.05, ** means p value of less than 0.01, and *** means p value of less than 0.001.

FIG. 4 shows the result of determining a decrease in the body weight of a mouse caused by a treatment with docetaxel, and the effect of suppressing the decrease in the body weight according to a combined treatment of docetaxel with the Phragmitis Rhizoma extract of the present invention. The control group (Control) represents a mouse not treated with any agent, and docetaxel+Phragmitis Rhizoma-125 and docetaxel+Phragmitis Rhizoma-250 mean a mouse received with combined treatment of docetaxel and the Phragmitis Rhizoma extract (125 μg/ml or 250 μg/ml, respectively).

FIG. 5 shows the result of determining a decrease in the number of mouse bone marrow cells caused by a treatment with docetaxel, and the effect of suppressing the decrease in the number of mouse bone marrow cells according to a combined treatment of docetaxel (15 nM) with the Phragmitis Rhizoma extract of the present invention at concentration of 125 μg/ml or 250 μg/ml. The control group represents a mouse not treated with any agent, and docetaxel+Phragmitis Rhizoma-125 and docetaxel+Phragmitis Rhizoma-250 mean a combined treatment of docetaxel and the Phragmitis Rhizoma extract (125 μg/ml or 250 μg/ml). In the figure, # indicates that the number of bone marrow cells after the treatment with docetaxel has a statistically significant difference compared to the control group which has not been treated with docetaxel in which p value is less than 0.05. * and ** indicate that there is a statistically significant difference in the number of bone marrow cells between the docetaxel treatment group and the group which has been treated with docetaxel and the Phragmitis Rhizoma extract in combination, in which * means p value of less than 0.05 and ** means p value of less than 0.01.

FIG. 6 shows an occurrence of abnormality in bone marrow structure of a mouse that is caused by a treatment with docetaxel (indicated with arrow) while such abnormality hardly occurs after the combined treatment of docetaxel with 125 mg/kg Phragmitis Rhizoma extract (Phragmitis Rhizoma-125) or 250 mg/kg Phragmitis Rhizoma extract (Phragmitis Rhizoma-250), in which the determination was made based on H&E staining.

FIG. 7 shows the result of histopathological analysis of thymus, in which tissue shrinkage and loss of functional lymphoid tissue according to administration of docetaxel are shown (indicated with arrow), and also the recovery from the tissue shrinkage and loss of functional lymphoid organs according to combined treatment of docetaxel with 125 mg/kg Phragmitis Rhizoma extract (Phragmitis Rhizoma-125) or 250 mg/kg Phragmitis Rhizoma extract (Phragmitis Rhizoma-250) is shown.

FIG. 8 shows the result of determining the property of Phragmitis Rhizoma extract to alleviate bone marrow suppression in an animal model which has been induced by a treatment with an anticancer agent. Docetaxel+Phragmitis Rhizoma-125 and docetaxel+Phragmitis Rhizoma-250 mean combined administration of docetaxel and Phragmitis Rhizoma 125 mg/kg and docetaxel and Phragmitis Rhizoma 250 mg/kg, respectively. * indicates that there is a statistically significant difference in IL-3 as an immune-stimulating cytokine between the docetaxel treatment group and the group which has been treated with docetaxel and the Phragmitis Rhizoma extract (125 mg/kg) in combination, with p value of less than 0.05.

FIG. 9 shows the result of determining a change in expression amount of the immune-stimulating cytokine in mouse spleen cells according to a treatment with the Phragmitis Rhizoma extract.

### BEST MODE(S) FOR CARRYING OUT THE INVENTION

The present invention relates to a composition containing Phragmitis Rhizoma extract as an active ingredient for prevention or treatment of a disorder caused by side effect of an anticancer agent.

The Phragmitis Rhizoma extract may be produced by a method comprising the following steps, but it is not limited thereto:

1) adding an extracting solvent to Phragmitis Rhizoma followed by extraction,

2) filtering an extract of the step 1), and

3) concentrating a filtered extract of the step 2) under reduced pressure followed by drying to product an extract.

The extracting solvent of the step 1) is preferably water, C₁-C₄ lower alcohol, or a mixture thereof, but it is not limited thereto.

With regard to the aforementioned production method, any kind of common methods that are generally known as an extraction method in the pertinent art, e.g., filtration, hot water extraction, impregnation extraction, extraction by reflux condensation, and ultrasonic extraction, can be used for obtaining a Phragmitis Rhizoma extract. It is preferable that the extraction is carried out by adding the extracting solvent in an amount of 1 to 20 times the volume of dried Phragmitis Rhizoma, and it is more preferably added in an amount of 3 to 10 times. The extraction temperature is preferably between 20° C. and 50° C., but it is not limited thereto. Furthermore, the extraction time is preferably between 10 hours and 100 hours, more preferably between 24 hours and 96 hours, and most preferably 72 hours, but it is not limited thereto. Regarding the above method, the concentration under reduced pressure of the step 3) is preferably carried out by using a vacuum condenser or a vacuum rotary evaporator, but it is not limited thereto. Furthermore, the drying is preferably carried out drying under reduced pressure, drying under vacuum, drying under boiling, spray drying, or freeze drying, but it is not limited thereto.

The cancer is preferably at least one selected from lung cancer, breast cancer, liver cancer, stomach cancer, colon cancer, colorectal cancer, skin cancer, bladder cancer, prostate cancer, ovary cancer, cervical cancer, thyroid cancer, kidney cancer, fibrosarcoma, melanoma, and leukemia, but it is not limited thereto and there is no limitation as long as it is diagnosable cancer.

The anticancer agent includes an antitumor antibiotic, a topoisomerase inhibitor, and a taxane-based anticancer agent, but it is not limited thereto. Any anticancer agent which can be clinically, pharmaceutically, and biomedically used is included.

The antitumor antibiotic is preferably at least one selected from a group consisting of actinomycin D, bleomycin sulfate, daunomycin, daunorubicin, doxorubicin, epirubicin, idarubicin, mitomycin, mitomycin-C, and mithramycin, but it is not limited thereto.

The topoisomerase inhibitor is preferably at least one selected from a group consisting of irinotecan, camptothecin, novobiocin, epirubicin, dactinomycin, amsacrine, teniposide, and etoposide, but it is not limited thereto.

The taxane-based anticancer agent is preferably one or both of paclitaxel and docetaxel.

The disorder caused by side effect is at least one selected from a group consisting of hematopoietic toxicity, anemia, and neutropenia, but it is not limited thereto.

The composition of the present invention contains, as an active ingredient, an extract or a fraction of Phragmitis Rhizoma of the present invention in an amount of 0.1 to 99.9% by weight relative to total weight of the composition, and it may also contain a pharmaceutically acceptable carrier, vehicle, or diluent.

The composition of the present invention may be prepared in various formulations including an oral formulation and a parenteral formulation. In case of producing a preparation, production is made by using a diluent or a vehicle such as filler, bulking agent, binding agent, moisturizing agent, disintegrating agent, or surfactant that are commonly used for producing a preparation. As for the solid preparation for oral administration, a tablet, a pill, a powder preparation, a granule, a capsule or the like are included, and such solid preparation is produced by mixing at least one compound with one or more vehicles such as starch, calcium carbonate, sucrose, lactose, or gelatin. Furthermore, other than simple vehicles, a lubricating agent such as magnesium stearate or talc is also used. As for the liquid preparation for oral administration, a suspension, a solution preparation for internal use, an emulsion, a syrup preparation, or the like can be mentioned. Other than water or liquid paraffin as a commonly used simple diluent, various kinds of a vehicle such as moisturizing agent, sweetening agent, aromatic agent, or preservatives may be included. Examples of a preparation for parenteral administration include a sterilized aqueous solution, a non-soluble agent, a suspension agent, an emulsion, a freeze-drying agent, and a suppository agent. As a water insoluble solvent or a suspending agent, propylene glycol, polyethylene glycol, or vegetable oil such as olive oil, and injectable ester such as ethylolate can be used. As a base for a suppository, witepsol, macrogol, tween 61, cacao fat, laurin fat, glycerogelatin, or the like can be used.

The composition of the present invention can be administered either orally or parenterally. In case of parenteral administration, it is preferable to choose external application on skin, intraperitoneal, rectal, intravenous, muscular, subcutaneous, endometrium injection, or intracerebroventricular injection. Most preferably, the composition is used for external application on skin.

The composition of the present invention is administered in a pharmaceutically effective amount. As described herein, the expression “pharmaceutically effective amount” means an amount sufficient for treating a disorder at reasonable benefit-risk ratio that can be applied for a medical treatment. The effective dose level may be determined based on a type or severeness of a disorder, activity of a pharmaceutical, sensitivity to a pharmaceutical, administration period, administration route, excretion ratio, time period for therapy, elements including a pharmaceutical used in combination, and other elements that are well known in the medical field. The composition of the present invention can be administered as a separate therapeutic agent, or it can be used in combination with other therapeutic agent. It can be administered in order or simultaneously with a conventional therapeutic agent. It can be also administered as single-dose or multi-dose. It is important to administer an amount which allows obtainment of the maximum effect with minimum dose while considering the all of the aforementioned elements without having any side effect, and the dosage can be easily determined by a person skilled in the pertinent art.

The dosage of the composition of the present invention may vary depending on body weight, age, sex, health state, diet of a patient, or administration period, administration method, excretion rate, and severity of disorder. However, the daily dosage is, in terms of the amount of the Phragmitis Rhizoma extract, 0.01 to 2,000 mg/kg, preferably 30 to 500 mg/kg, and more preferably 50 to 300 mg/kg, and it can be administered 1 to 6 times per day. The composition of the present invention may be used either singly, or in combination with surgical operation, radiation therapy, hormone therapy, chemotherapy, or therapy using biological response regulator.

The present invention also relates to a functional health food containing Phragmitis Rhizoma extract as an active ingredient for prevention or amelioration of a disorder caused by side effect of an anticancer agent. Extracting solvent for the Phragmitis Rhizoma extract is preferably water, C₁-C₄ lower alcohol, or a mixture thereof, but it is not limited thereto. The anticancer agent includes an antitumor antibiotic, a topoisomerase inhibitor, and a taxane-based anticancer agent, but it is not limited thereto. Any anticancer agent which can be clinically, pharmaceutically, and biomedically used is included. The disorder caused by side effect of an anticancer agent is characterized in that it is at least one selected from a group consisting of hematopoietic toxicity, anemia, and neutropenia.

To the health food of the present invention, the Phragmitis Rhizoma extract may be directly added. Alternatively, it may be used with other food or food ingredients, and suitably used according to a common method. Type of the health food is not particularly limited. Examples of the food to which the Phragmitis Rhizoma extract can be added include meat, sausage, bread, chocolate, candies, snacks, biscuits, pizza, ramen, other noodles, gums, dairy products including ice cream, various kinds of soup, beverage, tea, drink, alcohol beverage, and vitamin complex, and all health foods in general sense are included therein.

The health beverage containing the composition of the present invention may contain, like common beverages, various flavors or natural carbohydrates as an additional component. Examples of the natural carbohydrates include monosaccharides such as glucose or fructose, disaccharides such as maltose or sucrose, polysaccharides such as dextrin or cyclodextrin, and sugar alcohols such as xylitol, sorbitol, or erythritol. As a sweetening agent, a natural sweetening agent such as thaumatin or stevia extract or a synthetic sweetening agent such as saccharine or aspartame can be used. Ratio of the natural carbohydrates is generally about 0.01 to 0.04 g and preferably about 0.02 to 0.03 g relative to 100 g of the composition of the present invention.

Other than those active ingredients that are described above, the health food of the present invention may further contain various kinds of a nutritional agent, vitamins, electrolyte, flavors, a coloring agent, pectinic acid and salts thereof, alginic acid and salts thereof, organic acids, a protective colloid thickening agent, a pH adjusting agent, a stabilizing agent, a preservative, glycerin, alcohol, and a carbonating agent used for carbonate drink. In addition, fruit pulp for producing fruit juice or vegetable juice can be further contained. Those ingredients may be used either independently or in mixture. Ratio of those additives is not particularly critical. However, it is generally selected within a range of from 0.01 to 2 parts by weight relative to 100 parts by weight of the composition of the present invention.

The present invention also relates to anticancer adjuvant agent containing Phragmitis Rhizoma extract as an active ingredient. The anticancer adjuvant agent is characterized in that it suppresses at least one side effect of an anticancer agent selected from hematopoietic toxicity, anemia, and neutropenia that are induced by administration of an anticancer agent.

The anticancer adjuvant agent may additionally contain one or more active ingredients which exhibit a similar or the same activity as the Phragmitis Rhizoma extract. For clinical administration, the anticancer adjuvant agent can be administered either orally or parenterally. In case of parenteral administration, it can be administered by intraperitoneal injection, intrarectal injection, subcutaneous injection, intravenous injection, intramuscular injection, endometrium injection, intracerebroventricular injection, or intrathoracic injection. It may be also used in the form of a general pharmaceutical preparation.

The anticancer adjuvant agent may be used either singly, or in combination with surgical operation, radiation therapy, hormone therapy, chemotherapy, or therapy using biological response regulator. The daily dosage of the anticancer adjuvant agent is about 0.0001 to 100 mg/kg, and preferably 0.001 to 10 mg/kg, and it is administered either once or several divided times per day. It may vary depending on body weight, age, sex, health state, diet of a patient, administration period, administration method, excretion rate, and severity of disorder. For actual clinical administration, the anticancer adjuvant agent of the present invention can be administered as various parenteral formulations. In case of producing a preparation, production is made by using a diluent or a vehicle such as filler, bulking agent, binding agent, moisturizing agent, disintegrating agent, or surfactant that are commonly used for producing a preparation. As for the preparation for parenteral administration, sterilized aqueous solution, a non-aqueous preparation, a suspension, an emulsion, a freeze-dried preparation, and a suppository are included. As a non-aqueous solvent or a suspending agent, propylene glycol, polyethylene glycol, or vegetable oil such as olive oil, and injectable ester such as ethylolate can be used. As a base for a suppository, witepsol, macrogol, tween 61, cacao fat, laurin fat, glycerogelatin, or the like can be used.

Hereinbelow, the present invention is explained in greater detail in view of the Examples. However, the following Examples are given only for specific explanation of the present invention and it wound be evident to a person who has common knowledge in the pertinent art that the scope of the present invention is not limited by them.

### EXAMPLES

### Example 1. Preparation of Phragmitis Rhizoma Extract

To prepare a Phragmitis Rhizoma extract as an active ingredient of the present invention, Phragmitis Rhizoma was purchased from Kwangmyungdang Pharmaceuticals (Ulsan, South Korea). One hundred grams of the purchased Phragmitis Rhizoma were crushed, added to a round-bottomed flask, and mixed with 2 liters of water. By heating them in a water bath connected to a reflux extracting device which is equipped with a condenser, extraction was repeatedly carried out 2 times in total, 2 hours for each extraction. The obtained extract was subjected to filtration under reduced pressure by using a paper filter (Whatman No. 2) and a vacuum pump (GAST). By using a rotary evaporator (EYELA), the filtered liquid extract was concentrated under reduced pressure. The concentrated extract was then freeze-dried, and homogenized using a mortar and pestle to obtain Phragmitis Rhizoma extract. The extract was collected and sealed in a plastic container, and then stored in a 4° C. low temperature storage till to the test.

### Example 2. Determination of Hematopoietic Toxicity of Anticancer Agent

In order to determine the hematopoietic toxicity of an anticancer agent currently used for treating cancer patients, the influence of an anticancer agent at various concentrations on growth and differentiation of hematopoietic stem and progenitor cells in mouse bone marrow cells was determined based on an ex vivo test and an in vivo test. 1) Determination of hematopoietic toxicity of anticancer agent in mouse bone marrow cells (ex vivo)

Bone marrow cells were isolated from mouse femur. Subsequently, in a methocult GF M3434 medium containing recombinant murine stem cell factor (rm stem cell factor), rm TL-3, recombinant human IL-6 (rh IL-6), and rh erythropoietin to which docetaxel, doxorubicin, irinotecan, paclitaxel, or daunorubicin is added at various concentrations, the above mouse bone marrow cells were cultured for 7 to 10 days to induce the growth of hematopoietic stem and progenitor cells. The grown hematopoietic stem and progenitor cells were collected, and by carrying out CFU assay (i.e., Colony Forming Unit assay), the influence exhibited by an anticancer agent on growth and differentiation of the hematopoietic stem and progenitor cells was examined.

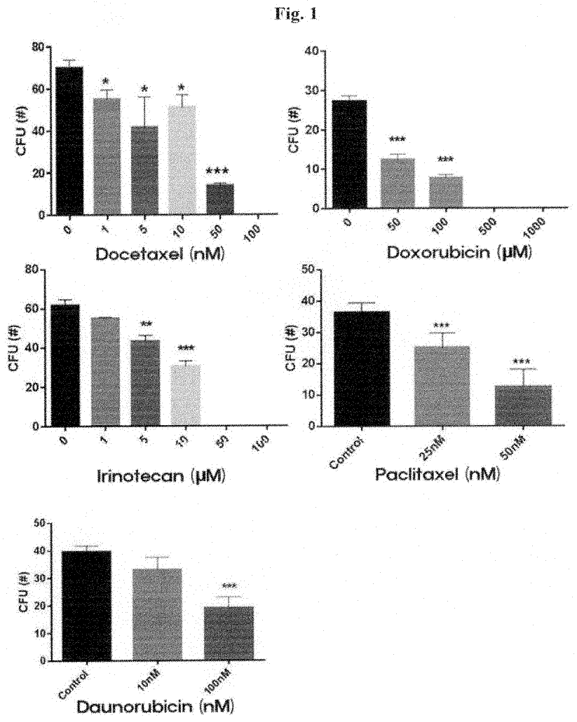

As a result, the hematopoietic toxicity was shown even at low concentrations as shown in FIG. 1. In particular, it was found that very significant hematopoietic toxicity is exhibited with docetaxel of 50 nM or higher, doxorubicin of 50 μM or higher, irinotecan of 5 μM or higher, paclitaxel of 25 nM or higher or daunorubicin of 100 nM or higher.

### 2) Determination of Hematopoietic Toxicity in Mouse Having Intraperitoneal Injection of Anticancer Agent (In Vivo)

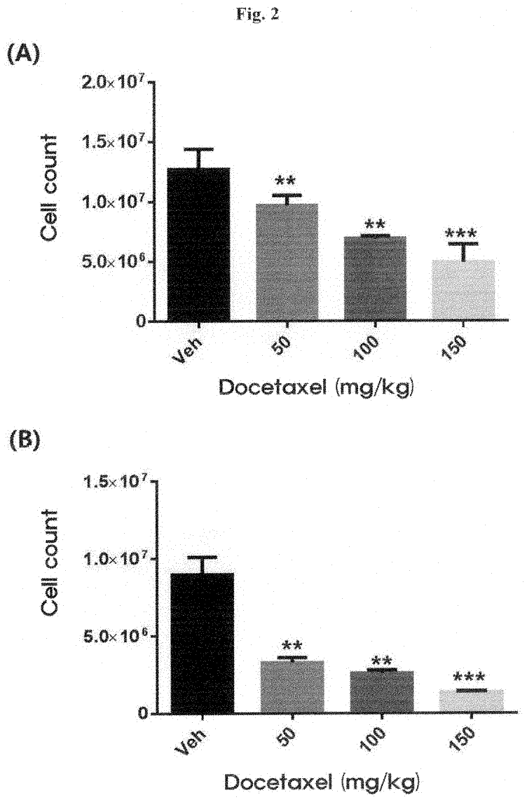

A C57BL/6 mouse was subjected to intraperitoneal injection of docetaxel at 50, 100, or 150 mg/kg. Three days and seven days after the injection, bone marrow cells were isolated from the mouse femur and counted, and the survival rate was measured. As a result, from any case of having docetaxel injection at 50, 100, or 150 mg/kg, a decrease in the number of the isolated bone marrow cells was observed in significant sense, three days and seven days after the injection, as shown in FIG. 2. Accordingly, it was confirmed that the hematopoietic toxicity is exhibited in an in vivo animal model.

### Example 3. Determination of Effect of Recovering Bone Marrow Toxicity by Phragmitis Rhizoma Extract Against Bone Marrow Toxicity Caused by Anticancer Agent

In order to determine whether or not the Phragmitis Rhizoma extract has an effect of ameliorating the hematopoietic toxicity that has been induced by an anticancer agent (i.e., 15 nM docetaxel, 100 nM doxorubicin, 100 μM irinotecan, 50 nM paclitaxel, or 100 nM daunorubicin) in bone marrow cells, a combined treatment of the Phragmitis Rhizoma extract with 15 nM docetaxel, 100 nM doxorubicin, 100 μM irinotecan, 50 nM paclitaxel, or 100 nM daunorubicin was carried out and the effect of ameliorating the hematopoietic toxicity was examined.

Specifically, bone marrow cells were separated from mouse femur, and then treated, in a methocult GF M3434 medium containing rm stem cell factor, rm IL-3, recombinant human IL-6, and rh erythropoietin, with an anticancer agent like 15 nM docetaxel as an agent inducing the hematopoietic toxicity in bone marrow cells. After that, the Phragmitis Rhizoma extract with concentration of 25, 50, or 100 μg/ml prepared in above Example 1 was added thereto, and growth of the hematopoietic stem and progenitor cells was induced according to culture for 7 days. After 7 days, the grown hematopoietic stem and progenitor cells were collected, and subjected to CFU assay to determine the growth of the hematopoietic stem and progenitor cells. As a negative control, the cells were treated with a solvent (i.e., PBS containing 0.5% DMSO) instead of the Phragmitis Rhizoma extract, and the same treatment as above was carried out.

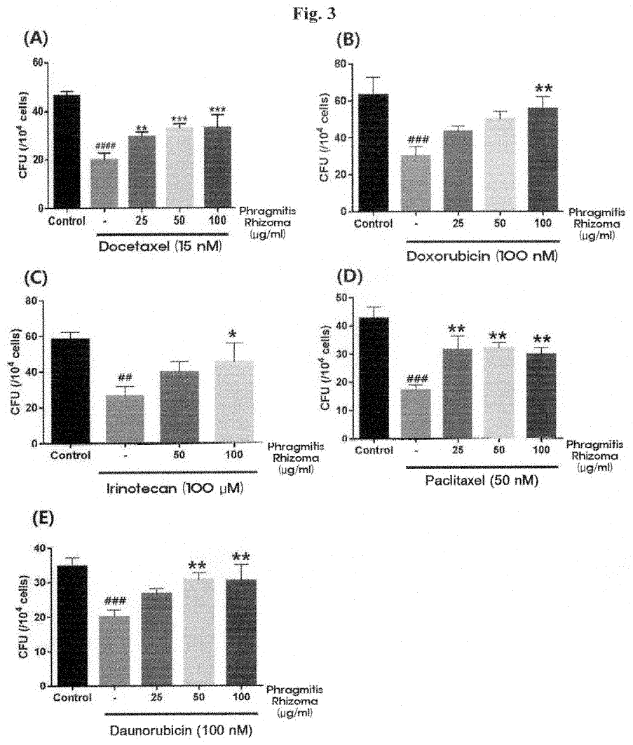

As a result, as shown in FIG. 3, it was confirmed that the decrease in colony of hematopoietic stem and progenitor cells, which has been induced by each anticancer agent, is recovered in a statistically significant sense by the Phragmitis Rhizoma extract (FIG. 3).

### Example 4. Determination of Alleviation of Bone Marrow Suppression by Phragmitis Rhizoma Extract in Animal Model Having Bone Marrow Suppression Induced by Docetaxel

In order to determine whether or not the Phragmitis Rhizoma extract exhibits an effect of ameliorating the hematopoietic toxicity in an animal model having bone marrow suppression, intraperitoneal injection of docetaxel to a mouse was carried out to induce bone marrow suppression. After that, a change brought by the Phragmitis Rhizoma extract was determined.

Specifically, C57BL/6 mice were categorized into 4 groups. Group 1 is a control, i.e., a group treated with saline containing 5% ethanol and 2% polysorbate 80. Group 2 is a docetaxel treatment group, i.e., a group with intraperitoneal injection (3 times) of 30 mg/kg docetaxel. Group 3 is a docetaxel and 125 mg/kg Phragmitis Rhizoma extract administration group, i.e., a group to which compulsory administration of 125 mg/kg Phragmitis Rhizoma extract was carried out, once a day starting from 2 days before the administration of docetaxel, and the compulsory administration of 125 mg/kg Phragmitis Rhizoma extract (once a day) was continued even for the 3 days of administering docetaxel. Lastly, Group 4 is a docetaxel and 250 mg/kg Phragmitis Rhizoma extract administration group, i.e., a group to which administration of 250 mg/kg Phragmitis Rhizoma extract was carried out, in the same manner as above Group 3, once a day starting from 2 days before the administration of docetaxel, and the compulsory administration of 250 mg/kg Phragmitis Rhizoma extract (once a day) was continued even for the 3 days of administering docetaxel.

After that, a change in body weight of the mouse belonging to each of the control group, docetaxel administration group, combined administration group with docetaxel and 125 mg/kg Phragmitis Rhizoma extract, and combined administration group with docetaxel and 250 mg/kg Phragmitis Rhizoma extract was measured every day. On Day 6, the mouse was sacrificed and the weight of the spleen and thymus, which play an important role in determining cell number change in bone marrow and hematopoiesis, was measured.

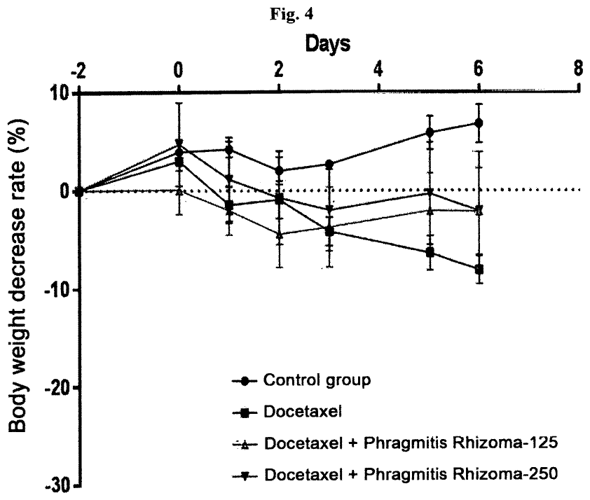

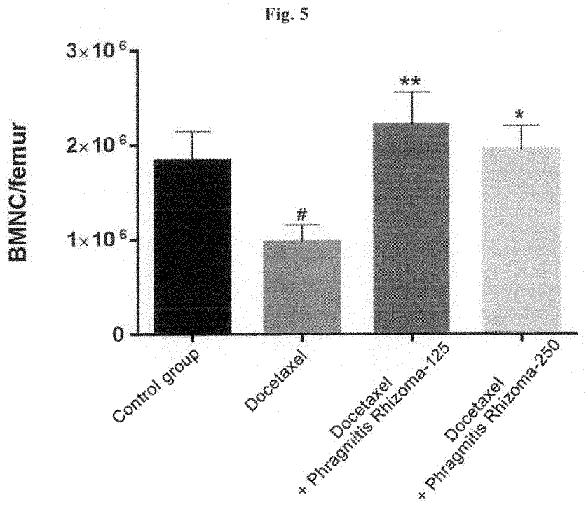

As a result, when the mouse was administered with docetaxel, a significant decrease in body weight was induced along with hematopoietic toxicity and bone marrow suppression as shown in FIG. 4 and FIG. 5. However, according to the combined administration of Phragmitis Rhizoma extract, the body weight decrease was alleviated (FIG. 4). Furthermore, while the number of bone marrow cells (i.e., bone marrow mononuclear cells) was decreased significantly in the docetaxel administration group compared to the control, recovery from the bone marrow suppression was observed from the group administered with the Phragmitis Rhizoma extract (FIG. 5).

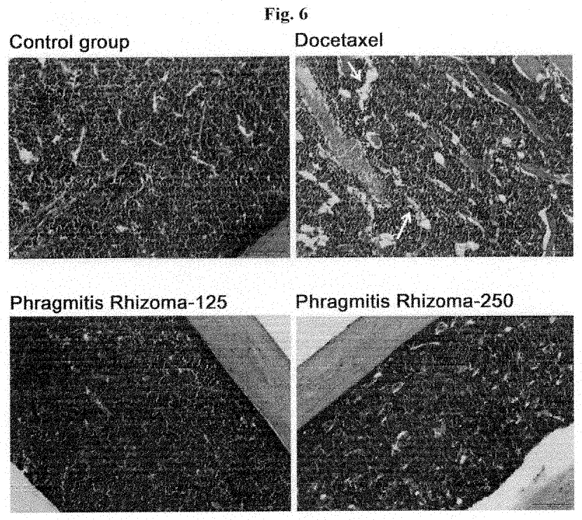

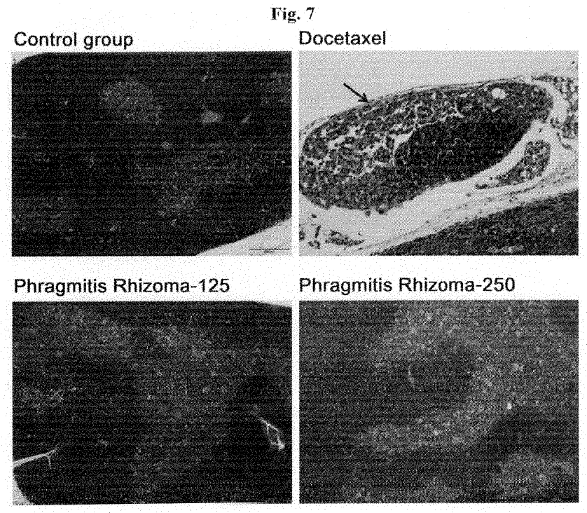

As a result of carrying out a histological analysis of a femur of mouse belonging to each administration group described above, it was found that the group administered with docetaxel only showed a hypocellularity and an abnormality in cellular structure in bone marrow (indicated with yellow arrow) as caused by a decrease in hematopoietic cells. However, in the combined administration group with Phragmitis Rhizoma extract, those phenomena were found to be rather diminished (FIG. 6). From the result of the histopathological analysis of the mouse thymus, it was also found that the organ shrinkage and loss of functional lymphoid organ, which have been caused by docetaxel administration, are recovered according to the combined administration of Phragmitis Rhizoma extract (FIG. 7).

Furthermore, in case of the mouse administered with docetaxel, an abnormality in the spleen and thymus as an organ involved with hematopoiesis was caused, thus showing a decrease in organ index as a result of a dramatic reduction in weight. However, it was found that the decrease in organ index is reversed in the group to which docetaxel and the Phragmitis Rhizoma extract are administered in combination.

Organ index (mg/g)=Weight of organ (mg)/Weight of animal (g)

From the group administered with docetaxel, shrinkage of spleen and thymus as an organ involved with the hematopoiesis, and weight decrease were observed. However, from the group administered with the Phragmitis Rhizoma extract, the effect of reversing such decrease was confirmed (Table 1).

TABLE 1
Organ index for determining change in weight of spleen
and thymus of animal belonging to docetaxel administration
group or combined administration group with docetaxel
and Phragmitis Rhizoma extract

| | Spleen index (mg/g) | Thymus index (mg/g) |
|---|---|---|
| Control group | 3.111 ± 0.58 | 2.360 ± 0.28 |
| Docetaxel | 2.366 ± 0.22 | 0.911 ± 0.22 |
| Docetaxel + Phragmitis | 3.004 ± 0.25 | 1.721 ± 0.07 |
| Rhizoma-125 | | |
| Docetaxel + Phragmitis | 2.842 ± 0.27 | 1.842 ± 0.49 |
| Rhizoma-250 | | |

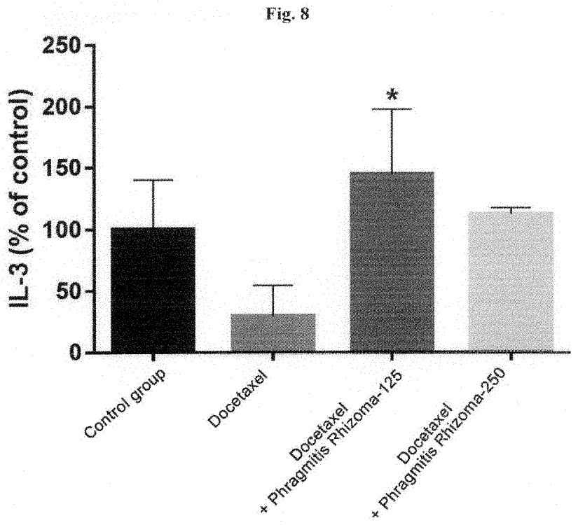

Meanwhile, from a mouse spleen tissue, mRNA was separated and a change in expression of cytokine, which is involved with the hematopoiesis, was determined. According to the administration of docetaxel, the hematopoiesis, in particular, expression of IL-3 deeply involved with differentiation and proliferation of myeloid progenitor cells, has largely decreased. On the contrary, according to combined administration of docetaxel and Phragmitis Rhizoma extract, the decreased IL-3 expression is recovered (FIG. 8).

### Example 5. Determination (In Vivo) of Influence of Phragmitis Rhizoma Extract on Immune-Stimulating Cytokine in Mouse Spleen Cells

In order to determine the influence of Phragmitis Rhizoma extract on expression of cytokine involved with hematopoiesis in an animal mold having bone marrow suppression, bone marrow suppression was induced by intraperitoneal injection of docetaxel to a mouse. After that, a change brought by the Phragmitis Rhizoma extract was determined.

Specifically, C57BL/6 mice were categorized into 5 groups. Group 1 is a control, i.e., a group treated with saline containing 5% ethanol and 2% polysorbate 80. Group 2 is a docetaxel treatment group, i.e., a group with intraperitoneal injection (3 times) of 30 mg/kg docetaxel. Group 3 is a docetaxel and 125 mg/kg Phragmitis Rhizoma extract administration group, i.e., a group to which compulsory administration of 125 mg/kg Phragmitis Rhizoma extract was carried out, once a day starting from 2 days before the administration of docetaxel, and the compulsory administration of 125 mg/kg Phragmitis Rhizoma extract (once a day) was continued even for the 3 days of administering docetaxel. Group 4 is a docetaxel and 250 mg/kg Phragmitis Rhizoma extract administration group, i.e., a group to which administration was carried out in the same manner as above Group 3. Lastly, Group 5 is a docetaxel and 500 mg/kg Phragmitis Rhizoma extract administration group, i.e., a group to which administration was carried out in the same manner as Group 3.

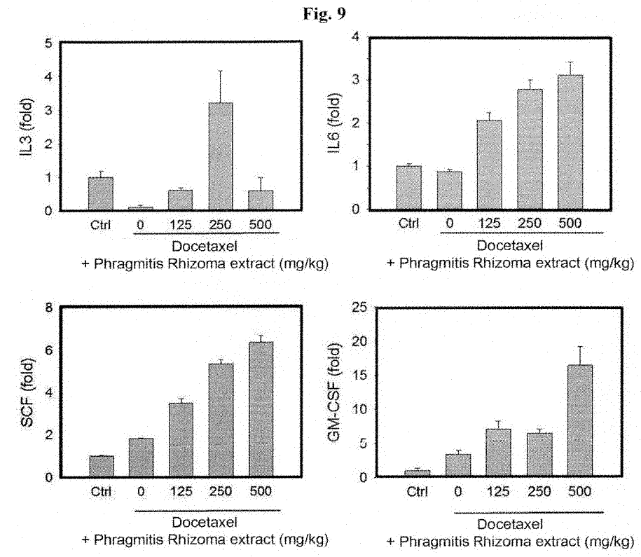

Spleen tissues were collected from the animal and mRNA was extracted therefrom. Then, as a result of determining a change in expression of cytokine involved with hematopoiesis, the increased expression of IL-3, IL-6, SCF (stem cell factor), and GM-CSF (granulocyte-macrophage colony-stimulating factor), which are the cytokines promoting the differentiation and proliferation of hematopoietic cells, was confirmed (FIG. 9).

### Example 6. Determination of Influence of Phragmitis Rhizoma Extract on Animal Model Having Docetaxel-Induced Bone Marrow Suppression

In order to determine the influence of Phragmitis Rhizoma extract on blood parameters of an animal model having bone marrow suppression, bone marrow suppression was induced by intraperitoneal injection of docetaxel to a mouse. After that, a change brought by the Phragmitis Rhizoma extract was determined.

Specifically, C57BL/6 mice were categorized into 5 groups. Group 1 is a control, i.e., a group treated with saline containing 5% ethanol and 2% polysorbate 80. Group 2 is a docetaxel treatment group, i.e., a group with intraperitoneal injection (3 times) of 30 mg/kg docetaxel. Group 3 is a docetaxel and 30 mg/kg Phragmitis Rhizoma extract administration group, i.e., a group to which compulsory administration of 30 mg/kg Phragmitis Rhizoma extract was carried out, once a day starting from 2 days before the administration of docetaxel, and the compulsory administration of 30 mg/kg Phragmitis Rhizoma extract (once a day) was continued even for the 3 days of administering docetaxel. Group 4 is a docetaxel and 100 mg/kg Phragmitis Rhizoma extract administration group, i.e., a group to which administration was carried out in the same manner as above Group 3. Lastly, Group 5 is a docetaxel and 300 mg/kg Phragmitis Rhizoma extract administration group, i.e., a group to which administration was carried out in the same manner as Group 3.

Blood was taken from the animal and a change in the number of blood cells was determined. As a result, a decrease in the number of the white blood cells, neutrophils, lymphocytes, and red blood cells was shown from the docetaxel administration group. However, a recovery from such decrease was obtained from the group administered with the Phragmitis Rhizoma extract in combination.

TABLE 2

| | WBCs | Neutrophils | Lymphocytes | RBCs |
|---|---|---|---|---|
| Control group | 3.44 ± 0.24 | 0.48 ± 0.02 | 2.88 ± 0.23 | 9.11 ± 0.18 |
| Docetaxel Group | 2.09 ± 0.19 | 0.30 ± 0.01 | 1.56 ± 0.22 | 7.72 ± 0.15 |
| Docetaxel + Phragmitis | 2.52 ± 0.15 | 0.47 ± 0.06^{##} | 1.83 ± 0.10 | 8.04 ± 0.13 |
| Rhizoma (30 mg/kg) | | | | |
| Docetaxel + Phragmitis | 2.59 ± 0.13^{#} | 0.41 ± 0.06 | 2.09 ± 0.10^{#} | 7.88 ± 0.13 |
| Rhizoma (100 mg/kg) | | | | |
| Docetaxel + Phragmitis | 2.84 ± 0.30 | 0.42 ± 0.07 | 2.22 ± 0.25 | 8.11 ± 0.18 |
| Rhizoma (300 mg/kg) | | | | |

The data of above Table 2 represent mean ± standard error (SEM) of the value obtained from ten mice, and ^{#} and ^{##} mean p < 0.05 and p < 0.01, respectively, vs. docetaxel administration group.

1-10. (canceled)

11: A method for preventing or treating a disorder caused by a side effect of an anticancer agent, the method comprising administering to a subject in need thereof a composition containing Phragmitis Rhizoma extract as an active ingredient.

12: The method of claim 11, wherein the extract is extracted by using water, C₁-C₄ lower alcohol, or a mixture thereof as a solvent.

13: The method of claim 11, wherein the anticancer agent is an antitumor antibiotic, a topoisomerase inhibitor, or a taxane-based anticancer agent.

14: The method of claim 13, wherein the anticancer agent is the antitumor antibiotic selected from the group consisting of actinomycin D, bleomycin sulfate, daunomycin, daunorubicin, doxorubicin, epirubicin, idarubicin, mitomycin, mitomycin-C, mithramycin, and a combination thereof.

15: The method of claim 13, wherein the anticancer agent is the topoisomerase inhibitor selected from the group consisting of irinotecan, camptothecin, novobiocin, epirubicin, dactinomycin, amsacrine, teniposide, etoposide, and a combination thereof.

16: The method of claim 13, wherein the anticancer agent is the taxane-based anticancer agent comprising at least one of paclitaxel, docetaxel and a combination thereof.

17: The method of claim 11, wherein the disorder caused by the side effect of the anticancer agent is at least one selected from the group consisting of hematopoietic toxicity, anemia, and neutropenia.

18: The method of claim 11, wherein the composition is included in a functional health food.

19: The method of claim 11, wherein the composition is an anticancer adjuvant agent.

20: The method of claim 19, wherein the disorder caused by the side effect of the anticancer agent is at least one selected from the group consisting of hematopoietic toxicity, anemia, and neutropenia.

21: The method of claim 11, wherein the subject has the disorder caused by the side effect of the anticancer agent, and the disorder is at least one selected from the group consisting of hematopoietic toxicity, anemia, and neutropenia.
